(19) United States
(12) Patent Application Publication
(10) Pub. No.: US 2019/0355457 A1
(43) Pub. Date: Nov. 21, 2019

(54) Telemedical apparatus, system, & method for providing medical services remotely

(71) Applicant: Bruce A. Lev, Sora (PA)

(72) Inventor: Bruce A. Lev, Sora (PA)

(21) Appl. No.: 12/387,948

(22) Filed: May 11, 2009

(51) Int. Cl. G16H 20/10; G16H 40/67

(52) U.S. Cl. CPC G16H 20/10; G16H 40/67

(57) ABSTRACT

A telemedical apparatus, system, and method for providing medical services remotely using electronic communications. The apparatus, system, and method includes a computer module having data input mechanisms on a user end, a data receiving and interpreting mechanism remotely located from the computer module, electronic communication devices between the computer module and the data receiving and interpreting mechanism, a mechanism for relaxing a user prior to and during use, a mechanism for providing medical services after the user's data and information is entered and processed by the data receiving and interpreting mechanism, and a mechanism for sending, receiving, and filling medical prescriptions.

### FIELD OF INVENTION

This invention relates to telemedical apparatuses, systems, and methods for providing medical services remotely using electronic communications and equipment, biological vital sign and personal information, and available medical practitioners.

### BACKGROUND OF THE INVENTION

There are numerous instances and situations when a person needs medical attention and care but is either too far away from such services and providers or the attention and care needed is not urgent and can be serviced remotely and conveniently using an apparatus, system, and method as described herein.

In the past, a person needing medical attention and care would need to schedule an appointment with a practitioner, commute to that practitioner's location, wait in a waiting room for a period of time, and fill in forms and paperwork by hand before receiving even a basic examination. It became obvious that a need existed to incorporate available technologies into an apparatus, system, and method that could provide these services remotely and electronically.

Further, it has been proven in numerous medical tests, trials, and actual cases that a person in a calm or relaxed state will display more accurate and informative vital signs than those that are agitated, tired, anxious, frustrated, confused, etc. When a user first arrives at a telemedical apparatus of the prior art they may be unsure and anxious as to what to expect from the apparatus or the lack thereof of a professional doctor or nurse being there in person to offer a personal touch, they may be agitated by their previous daily events, they may be tired by their commute to the telemedical module, etc. Therefore, it is extremely important that vital signs taken remotely be preceded by a mechanism, means, or method for relaxation to thereby improve the quality and accuracy of the vital signs retrieved, the analyses calculated, and the advice and services thereby rendered.

Within the configuration of the instant invention, a chosen relaxation mechanism, means, or method accompanies and communicates with the computer module in a convenient location for a user to connect with quickly, easily, and comfortably. This relaxation mechanism, means, or method can be formed in many configurations, such as providing a comfortable chair or recliner, hand vibrators or warmers that can be attached to each vertical side of the module, a head set that vibrates and cools a user's head, an odorizer that produces a pleasant smell, a noise generator that plays pleasant tones or music, offering meditation techniques, offering progressive neuromuscular relaxation techniques, offering message therapy, offering medicinal therapy, offering hypnosis techniques and methods; providing a misting mechanism; providing a water tank for a user to lay and relax in, providing biofeedback mechanisms, or similar means, mechanisms, or methods, or a combination of any thereof.

Another deficiency of current telemedical designs and apparatuses is that once a user has been diagnosed and medical treatment and services been determined there is no convenient mechanism, means, or method to deliver a prescription to the remote user nor to fill the prescription by that user. The instant invention overcomes these deficiencies by providing a mechanism for receiving and printing a prescription which can then be carried by the user to a convenient or local pharmacy to fill. The mechanism for receiving and printing a prescription can be formed as a combination of facsimile software within the computer module and a small printing mechanism communicating with the computer module. The mechanism can also be formed using digital files created by a medical professional and sent electronically to the computer module to be stored or printed. Or any equivalent or obvious variations thereof. Therefore, a prescription can be written by a medical professional, sent electronically to the computer module, printed by the printing mechanism, obtained by the user, taken by the user to a convenient or local pharmacy, and filled by said pharmacy.

A variation of the prescription filling mechanism, means, or method would be to have a chosen pharmacy or pharmacies integrated within the telemedical system that could be adapted and capable of receiving the user's determined prescription directly and then filling that prescription for the user. The user could then either pick up the prescription in person or have the prescription delivered to a desired location chosen.

Other apparatuses, systems, and methods have been developed to accomplish some of these telemedical functions and requirements, including the system of Echerer, U.S. Pat. No. 6,046,761. The system of Echerer does include components that do accomplish basic telemedical functions. However, there are no mechanisms, means, or methods within the Echerer system that will relax a user or better well prepare them for the reading of their vital signs. As such the readings taken may not be accurate and the medical advice and services rendered thereof may not be accurate or helpful. In fact, the numerous steps that Echerer uses before taking the user's vital signs, for example, verifying the identity of the user by asking them to hold up an identification card to the system's video camera and determining an acceptable mode of payment, may actually increase the user's stress levels and cause further inaccuracies within the readings of their vital signs.

Another improvement over the system of Echerer that the instant invention offers is that it does not rely on “a means for verifying the identity of said patient” or “means for establishing a mode of payment” as claimed by Echerer. Many times a user does not have time or the desire to create an on-line identity or verify his identity before sending their vital information and receiving attention and care, let alone establishing a mode of payment. The instant invention operates without these requirements and thereby offers faster and better attention and care to a user.

Further, the system of Echerer does not address the problem of sending, receiving, and filling necessary prescriptions for its users.

Another apparatus, system, and method was set forth by Sjoquist, U.S. Pat. No. 6,409,660. This system also does include components that do accomplish basic telemedical functions. However, once again, there are no mechanisms, means, or methods within the Sjoquist system that will relax a user or better well prepare them for the reading of their vital signs. As such the readings taken may not be accurate and the medical advice and services rendered thereof may not be accurate or helpful.

Another improvement over Sjoquist that the instant invention offers is that it does not rely on “a means for assigning priorities to different types of information upon transmission of data to a central unit” as claimed by Sjoquist. The data receiving and interpreting mechanism on the receiving end of the instant invention retrieves and processes all the information sent by the user in an extremely fast and efficient manner no matter how much data is sent at the same time or in what order, thereby negating the need to “prioritize” the information first and actually saving time and avoiding a degree of possible computer error.

Further, the system of Sjoquist also does not address the problem of sending, receiving, and filling necessary prescriptions for its users.

### SUMMARY OF THE INVENTION

The instant invention is set forth as a telemedical apparatus, system, and method for providing medical services remotely using electronic communications and equipment, biological vital sign and personal information, and available medical practitioners.

The “system” for providing medical services remotely includes the use of electronic communications and equipment, including but not limited to the use of internet technology, the input and interpreting of biological vital sign and personal information, and the transfer of advice and medical services by available medical practitioners.

The “apparatus” for providing medical services remotely includes a computer module having a data input mechanism on a user end, a data receiving and interpreting mechanism on the receiving end, electronic communication devices between the computer module and the data receiving and interpreting unit, a mechanism for relaxing a user prior to and during use, and a mechanism for providing medical services after the user's data and information is processed by the data receiving and interpreting unit, including sending, receiving, and filling necessary prescriptions for its users.

The computer module incorporates advanced software packages and touch-screen capabilities. Other components are then removably connected to the computer module either by wires or wirelessly that perform functions such as measuring a user's blood pressure, weight, cholesterol, pulse, temperature, and glucose level. These components may also include a spirometer for monitoring and diagnosing asthma systems, a diagnostic camera for up close visual inspections, including dermatological evaluations, and even radiological mechanisms to perform the tests and functions of a radiologist. The computer module is also designed to receive additional information and data from a user through an input mechanism, which includes a series of questions that said user responds to and inputs their answers.

Before the biological vital readings are taken from the user, any one or a combination of a series of stress reducing mechanisms and techniques are used to relax the user so that their vital signs become more steady and accurate. These mechanisms and techniques may include the use of a chair or recliner, hand vibrators or warmers that can be attached to each vertical side of the module, a head set that vibrates and cools a user's head, an odorizer that produces a pleasant smell, a noise generator that plays pleasant tones or music, meditation techniques, progressive neuromuscular relaxation techniques, message therapy, medicinal therapy, hypnosis, a misting mechanism, laying in a water tank, biofeedback mechanisms, or any similar means, mechanisms, or methods, or a combination of any thereof.

Once the user is sufficiently relaxed they are appropriately connected to the computer module with respective medical devices and their biological vital signs and necessary information are retrieved. The computer module then connects electronically with the data receiving and interpreting mechanism on the receiving end. The data receiving and interpreting mechanism can include trained personnel interfacing with the user, and computer modules with access to a large data base that includes previous patient information, a library of medical information, automated medical diagnostic tools, and a listing of medical practitioners. Once all the necessary information and vital signs are processed and an initial diagnosis is accomplished, the data receiving and interpreting mechanism then connects the user with an appropriate medical practitioner for further instructions, attention, and medical services. One of the medical services that can be provided includes writing a prescription for medical treatment, wherein said prescription is forwarded to the computer module on the user end and is thereby adapted and available to be used by the user to obtain the prescription at a pharmacy that is convenient for the user.

Finally, the “method” for providing medical services remotely includes providing the apparatus as set forth herein, including providing a means for relaxation, providing a means for data receiving and interpreting, providing medical expertise and services by medical practitioners, and providing means for sending and filling medical prescriptions.

### BRIEF DESCRIPTION OF THE DRAWINGS

FIG. 1 is a perspective view of the telemedical apparatus on the user end including the computer module, mechanisms for relaxation, and the mechanism for receiving and printing prescriptions.

FIG. 2 is a flow chart showing the preferred interrelationships between portions of the telemedical system and method.

FIG. 3 is a perspective view of a water tank being used as the mechanism for relaxation.

FIG. 4 is a perspective view of an example of a data receiving and interpreting mechanism formed as a remotely located call center.

FIG. 5 is a view of a remotely located medical practitioner including a mechanism for sending prescriptions electronically.

FIG. 6 is a perspective view of a user visiting a pharmacy to have their prescription filled.

FIG. 7 is a perspective view of the means for relaxation formed as hand warmers and vibrators.

FIG. 8 is a perspective view of the means for relaxation formed as a head set that vibrates and cools a user's head.

### DESCRIPTION OF THE PREFERRED EMBODIMENT

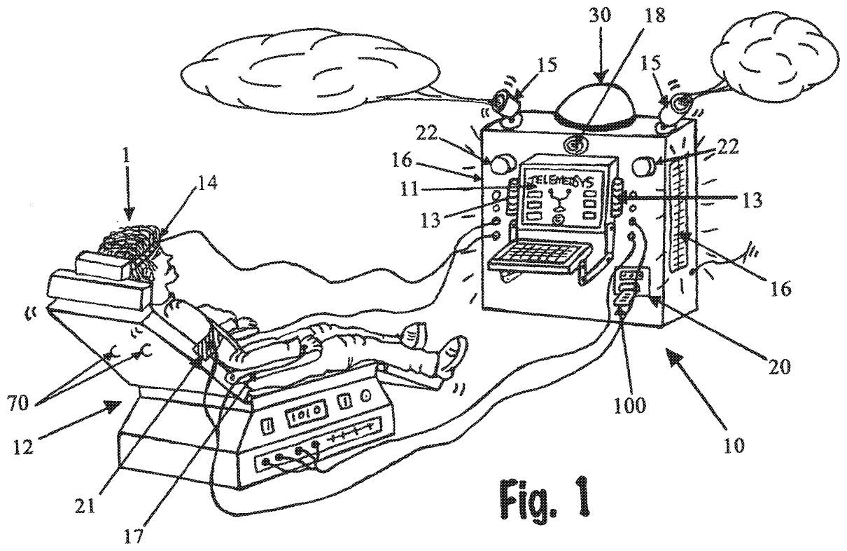

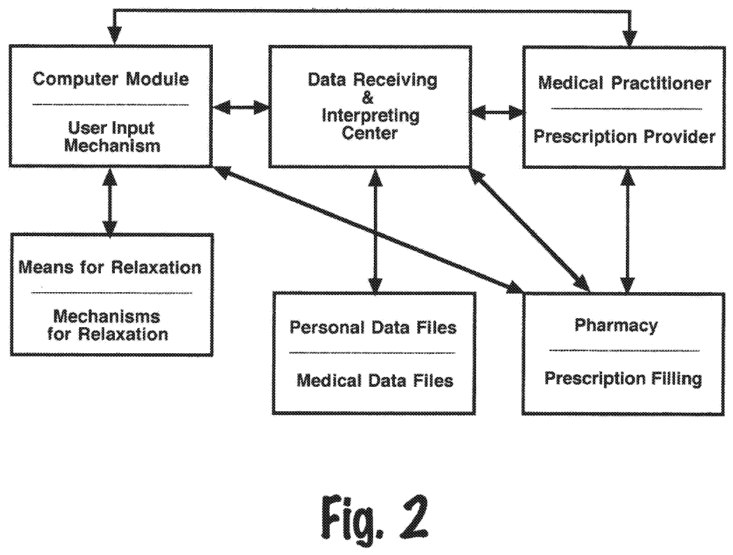

The preferred embodiment of the instant invention is illustrated in FIGS. 1-8 as a telemedical apparatus, system, and method.

FIG. 1 illustrates a perspective view of the telemedical apparatus on the user end including the computer module 10, mechanisms for relaxation described in further detail below, and the mechanism for receiving and printing prescriptions 20. The computer module incorporates many of the modern technical equipment, including advanced software packages, touch-screen technology, and electronic communications technology, including wireless internet sending and receiving mechanisms within member 30.

When a user 1 first approaches the telemedical system computer module they simply touch the computer module screen 11 where it displays the instruction to do so, such as a “Press here to begin” image. The user will then be greeted by voice and/or visual commands to guide them through a process that includes the input of that user's necessary personal and vital sign information using the computer module touch screen capabilities or optional keyboard, and selected vital sign retrieval mechanisms, described in further detail below.

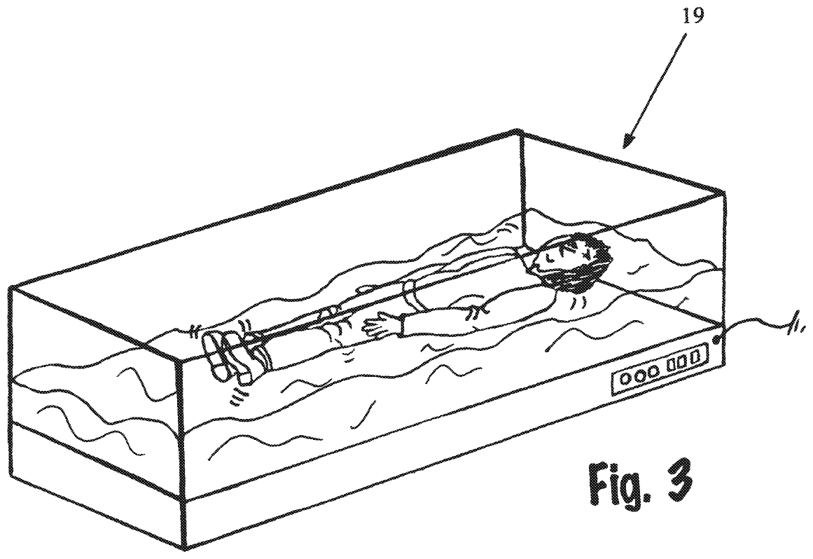

Once the user is logged in and ready they can choose between any one or a combination of a series of the stress reducing mechanisms and techniques offered and used to relax the user so that their vital signs become more steady and accurate. These mechanisms and techniques include the use of a chair or recliner 12, hand vibrators and warmers 13 that can be releasably attached to each vertical side of the computer module screen or casing, a head set 14 that vibrates and cools a user's head, odorizing and misting members 15 that produces pleasant smells and mist, noise generators 16 that plays pleasant tones or music, meditation techniques, progressive neuromuscular relaxation techniques, message therapy which can be accomplished by vibrating the chair or recliner and incorporating rollers 70 therein, or even by having a trained specialist apply manual techniques, medicinal therapy 17, hypnosis, which can be accomplished visually using mechanism 18 that spins with a hypnotic pattern, or by having a trained specialist apply other techniques, providing a water tank 19, as shown in FIG. 3, for a user to lay and float in, biofeedback mechanisms, any similar means, mechanisms, or methods, or a combination of any thereof.

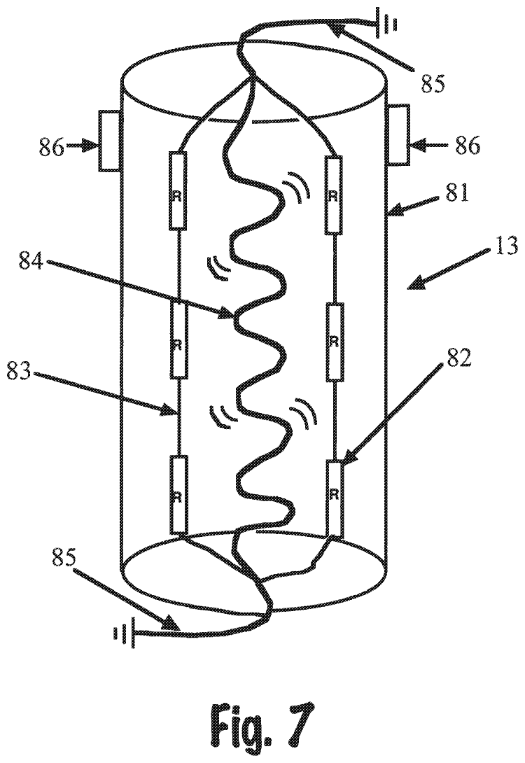

A more detailed illustration of the hand vibrators and warmers 13 is set forth in FIG. 7. In this embodiment the hand vibrators and warmers are formed having hollow cylindrical bodies 81. Within and attached to the inner walls of the cylindrical bodies are resistor members 82, which are individually marked each with an R and are interconnected by conducting wires 83, that heat up when a current is passed therethrough. Further, a vibrating member 84, which in this embodiment is formed as a spring type member, passes through the middle of the body and vibrates when a current is also passed therethrough. The hand vibrators and warmers are electrically connected to the computer module via electric conductor members 85, and releasably mechanically connected to the computer module via connector members 86. When the user chooses this means for relaxation, they place one hand on each member 13. The computer module then send an electric current through the wires to heat and vibrate the user's hands until the user is sufficiently relaxed.

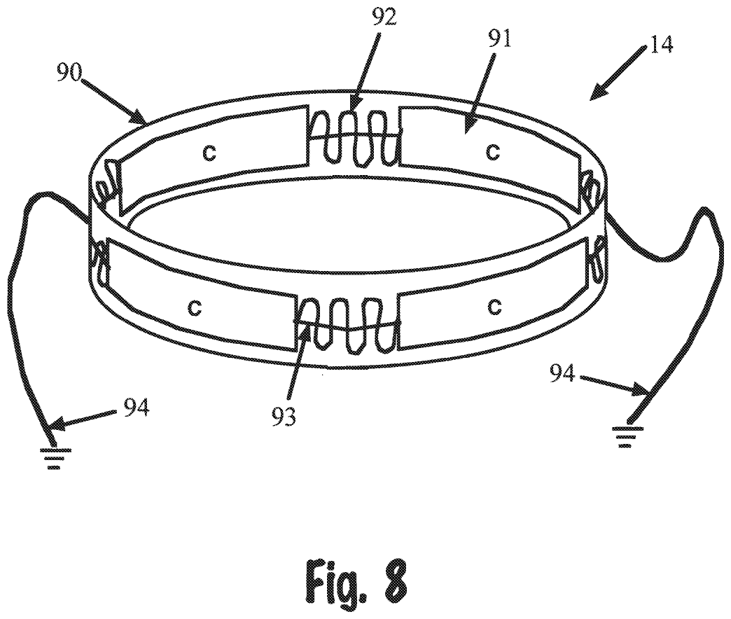

A more detailed illustration of the head set 14 that vibrates and cools a user's head is set forth in FIG. 8. In this embodiment the head set is formed as a circular band 90 having condenser cooling members 91 interconnected by electrical members 93, and vibrator members 92 interconnected between the condenser cooling members and attached to the inner side thereof. The condenser cooling members and vibrators are electrically connected to the computer module via electric conductor members 94. When the user chooses this means for relaxation, they place the head set on their head. The computer module then send an electric current through the wires to the cooling and vibrating members until the user is sufficiently relaxed.

A meditation technique, a progressive neuromuscular relaxation technique, and a hypnosis technique are also being employed and used by user 1 and illustrated in FIG. 1. Therefore, user 1 is in a completely relaxed state for their biological vital signs to be taken.

Once the user is ready for vital sign entry, the vital sign retrieving components are connected to the user, including in no particular order, mechanisms to measure and record blood pressure 21, weight, cholesterol, pulse rate, temperature, and glucose level. These components can also include a spirometer for monitoring and diagnosing asthma systems, three-dimensional diagnostic cameras 22 for up close visual inspections, including dermatological evaluations, and even radiological mechanisms.

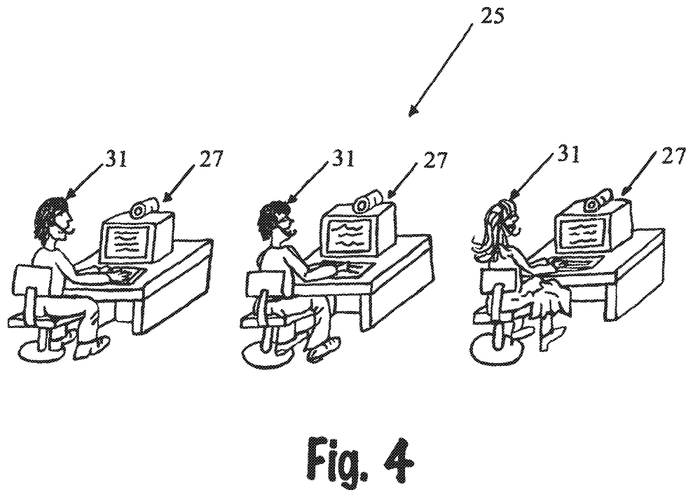

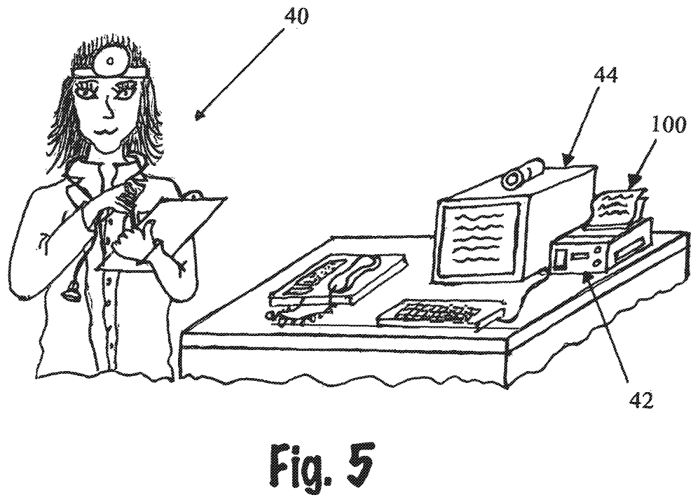

Once the user properly connects to the module and their vital signs and necessary information have been retrieved, the computer module connects electronically, usually via the internet, with a data receiving and interpreting mechanism 25 on the receiving end, as illustrated in FIG. 4. This data receiving and interpreting mechanism includes trained personnel 31 interfacing with the user, and computer modules 27 with access to a large data base that includes previous patient information, a library of medical information, automated medical diagnostic tools, and a listing of medical practitioners. Once all the necessary information and vital signs are processed and an initial diagnosis is accomplished, the data receiving and interpreting mechanism then connects the user to the appropriate medical practitioner 40, as illustrated in FIG. 5, for further instructions, attention, and medical services. The medical practitioner has the ability and means to research, diagnose, and write and send prescriptions to either the user directly or through the data receiving and interpreting mechanism. As shown in FIG. 5, medical practitioner 40 can use a scanning and/or facsimile device 42 to retrieve and send the necessary prescription, or create a computer file and send the prescription using prescription software, computer module 44, and internet technology, or any variation or combination of known technologies.

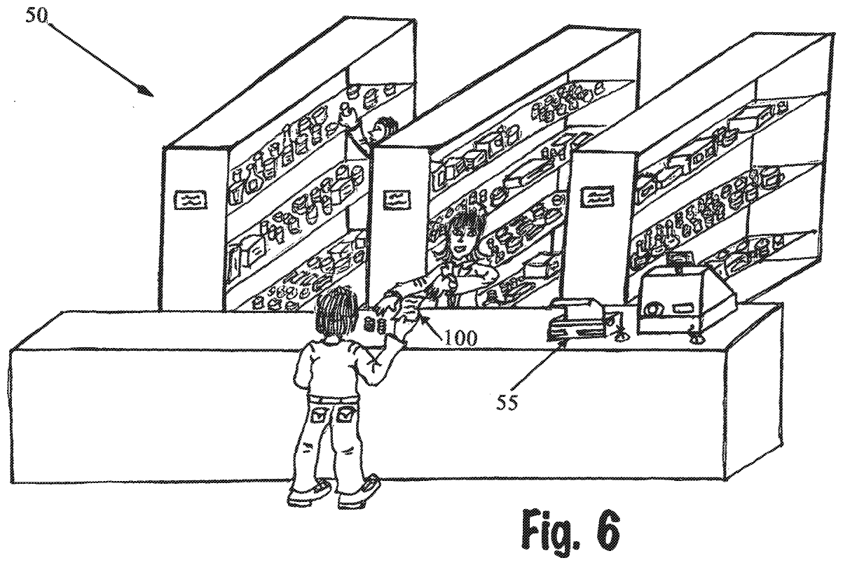

One of the most important medical services provided within this apparatus, system, and method of providing medical services remotely includes a mechanism for receiving and printing a prescription, illustrated in FIG. 1 as member 20. The prescription, viewed in FIGS. 1, 5, and 6 as member 100, can then be carried by the user to a convenient and local pharmacy 50 to fill, as illustrated in FIG. 6. The mechanism can be formed as a combination of facsimile software within the computer module and a small printing mechanism 20 communicating with the computer module. The mechanism can be also formed as digital files created by a medical professional and sent electronically to the computer module to be stored or printed. Or any equivalent or obvious variations thereof. Therefore, a prescription can be written by a medical professional, sent electronically to the computer module, printed by the printing mechanism, obtained by the user, taken by the user to a convenient and local pharmacy, and filled by said pharmacy.

A variation of the prescription filling apparatus, means, or method would be to have a chosen pharmacy 50 or pharmacies integrated within the telemedical system that could be adapted and capable of receiving the user's determined prescription directly, either using computer files or facsimile technology and a printer, for example, member 55 in FIG. 6, and then filling that prescription for the user. The user could either pick up the prescription in person or have the prescription delivered to a chosen location.

All previous relevant prior art, and any and all other obvious modifications to one or more of the parts, mechanisms, systems, or methods of this invention are inherently disclosed and incorporated herein.

What is claimed is:

1. A telemedical apparatus comprising a computer module; a data receiving and interpreting mechanism remotely located from said computer module; an electronic communication mechanism between and communicating with said computer module and said data receiving and interpreting mechanism; a mechanism for relaxation; and a mechanism for providing medical advice and services after data and information is sent by said computer module to and is processed by said data receiving and interpreting mechanism.

2. The telemedical apparatus of claim 1, further comprising a mechanism for receiving and printing medical prescriptions from a medical practitioner.

3. The telemedical apparatus of claim 2, wherein said mechanism for receiving and printing medical prescriptions comprises software within said computer module and a printing mechanism communicating with said computer module for printing said medical prescriptions.

4. The telemedical apparatus of claim 1, wherein said computer module is adapted to have medical components communicating therewith for obtaining, storing, and sending biological vital signs, including blood pressure, weight, cholesterol, pulse rate, temperature, and glucose levels; and wherein medical components are adapted to communicate with said computer module, said components chosen from a list of components including a blood pressure measuring device, a weight measuring device, a cholesterol measuring device, a pulse rate measuring device, a temperature measuring device, a glucose level measuring device, a spirometer for monitoring and diagnosing asthma systems, three-dimensional diagnostic cameras for up close visual inspections, and radiological devices.

5. The telemedical apparatus of claim 1, wherein said data receiving and interpreting mechanism comprises computers and trained personnel monitoring said computers; wherein said mechanism for providing medical advice and services includes evaluating said data and information sent thereto and determining which type of medical practitioner needs to be contacted, contacting said medical practitioner, forwarding said data and information to said practitioner, receiving a response from said practitioner, forwarding said response to said computer module; and wherein said response from said medical practitioner includes determining a medical condition and providing a medical prescription for medical treatment, said medical prescription is then forwarded to said computer module and is thereby adapted and available to be used to obtain said medical prescription by the person using said computer module.

6. The telemedical apparatus of claim 1, wherein said mechanism for relaxation is adjustable.

7. The telemedical apparatus of claim 6, wherein said mechanism for relaxation comprises a seat member; a reclining back rest member pivotably and adjustably attached to said seat member; a foot rest member pivotably and adjustably attached to said seat member; and a head rest member pivotably and adjustably attached to said back rest member.

8. The telemedical apparatus of claim 1, wherein said mechanism for relaxation comprises hand vibrators and warmers that are adapted to be attached to each vertical side of said computer module.

9. The telemedical apparatus of claim 1, wherein said mechanism for relaxation comprises a head set that includes vibrators and cooling elements for vibrating and cooling a user's head.

10. The telemedical apparatus of claim 1, wherein said mechanism for relaxation comprises an odorizing and misting member that produces pleasant smells and mist; and a noise generating member that plays pleasant tones and music.

11. The telemedical apparatus of claim 1, wherein said mechanism for relaxation includes the use of meditation techniques.

12. The telemedical apparatus of claim 1, wherein said mechanism for relaxation includes medicinal therapy.

13. The telemedical apparatus of claim 1, wherein said mechanism for relaxation includes means for hypnotizing.

14. The telemedical apparatus of claim 1, wherein said mechanism for relaxation includes a water tank adapted for a user to lay and float in.

15. The telemedical apparatus of claim 1, wherein said electronic communication mechanism includes the use of technologies from a list of technologies including internet, radio, telephony, teletype, and facsimile.

16. A telemedical system comprising user input means; data receiving and interpreting means remotely located from said user input means; electronic communication means between said user input means and said data receiving and interpreting means; means for relaxation; and a means for providing medical advice and services after data and information is sent by said user input means to said data receiving and interpreting means and is processed by said data receiving and interpreting means, such that a user can locate said input means, use said means for relaxation to stabilize their biological vital signs, use said input means to collect and send their personal information and stabilized vital signs to said data receiving and interpreting means, have their personal information and stabilized vital signs interpreted by said data receiving and interpreting means, and then receive medical advice and services from said means for providing medical advice and services.

17. The telemedical system of claim 16, further comprising means for receiving and printing medical prescriptions sent by said means for providing medical advice and services.

18. The telemedical system of claim 16, wherein said data receiving and interpreting mechanism includes computers and trained personnel monitoring said computers; and wherein said mechanism for providing medical advice and services includes evaluating said data and information sent thereto and determining which type of medical practitioner needs to be contacted, contacting said medical practitioner, forwarding said data and information to said practitioner, receiving a response from said practitioner, and forwarding said response to said computer module; and wherein said response from said medical practitioner includes determining a medical condition and providing a medical prescription for medical treatment, said medical prescription is then forwarded to said computer module and is thereby adapted and available to be used to obtain said medical prescription by the person using said computer module.

19. A method of providing medical services remotely using electronic communications comprising the steps of providing user input means; providing data receiving and interpreting means remotely located from said user input means; providing electronic communication means between said user input means and said data receiving and interpreting means; providing means for providing medical advice and services after data and information is sent by said user input means to said data receiving and interpreting means and is processed by said data receiving and interpreting means; and providing means for receiving and printing prescriptions, such that a user can easily and quickly locate said user input means, have their information and vital signs taken and inputted, have their information and vital signs sent to said data receiving and interpreting means, have their information and vital signs interpreted and used to locate and connect with an appropriate medical services provider, have the medical services provider suggest treatments and send relevant prescriptions and directions back through the electronic communication means to the user, which is then adapted and available to be used to obtain said prescriptions by said user.

20. The method of providing medical services remotely using electronic communications of claim 19, further comprising the step of providing means for relaxation for helping a user become relaxed and ready for their biological vital signs to be measured and retrieved.

21. A telemedical apparatus comprising a computer module; a data receiving and interpreting mechanism remotely located from said computer module; an electronic communication mechanism between and communicating with said computer module and said data receiving and interpreting mechanism; and a mechanism for providing medical advice and services after data and information is sent by said computer module to and is processed by said data receiving and interpreting mechanism; and a mechanism for receiving and printing medical prescriptions from a medical practitioner.

22. The telemedical apparatus of claim 21, wherein said mechanism for receiving and printing medical prescriptions comprises software within said computer module and a printing mechanism communicating with said computer module for printing said medical prescriptions.

23. The telemedical apparatus of claim 21, further comprising a mechanism for relaxation.

24. The telemedical apparatus of claim 23, wherein said mechanism for relaxation comprises a seat member; a reclining back rest member pivotably and adjustably attached to said seat member; a foot rest member pivotably and adjustably attached to said seat member; and a head rest member pivotably and adjustably attached to said back rest member; and a noise generating member that plays pleasant tones and music.
